(19) United States
(12) Patent Application Publication
(10) Pub. No.: US 2007/0149480 A1
(43) Pub. Date: Jun. 28, 2007

(54) PHARMACEUTICAL COMPOSITION FOR DELIVERY OF RECEPTOR TYROSINE KINASE INHIBITING (RTKi) COMPOUNDS TO THE EYE

(75) Inventors: Malay Ghosh, Fort Worth, TX (US); Wesley Wehsin Han, Arlington, TX (US); Way-Yu Lin, Arlington, TX (US)

Correspondence Address: ALCON, IP LEGAL, TB4-8, 6201 SOUTH FREEWAY, FORT WORTH, TX, 76134, US

(73) Assignee: Alcon, Inc., Fort Worth, TX (US)

(21) Appl. No.: 11/614,804

(22) Filed: Dec. 21, 2006

Related U.S. Application Data
(60) Provisional application No. 60/753,642, filed on Dec. 23, 2005.

(51) Int. Cl. A61K 31/724; A61K 31/416

(52) U.S. Cl. 514/58; 514/406

(57) ABSTRACT

The present invention relates to development of efficacious pharmaceutical compositions comprising an anti-angiogenic compound in a therapeutically effective amount complexed with or encapsulated in a cyclodextrin derivative.

This application claims priority to U.S. application Ser. No. 60/753,642, filed Dec. 23, 2005.

### BACKGROUND OF THE INVENTION

1. Field of the Invention

The present invention relates to unique compositions containing compounds with poor solubility and methods useful for treating pathological states that arise or are exacerbated by ocular angiogenesis, inflammation and vascular leakage such as AMD, DR, diabetic macular edema etc., and more specifically, to compositions containing agent with anti-angiogenic, anti-inflammatory or anti-vascular permeability property for use in treating ocular disorders.

2. Description of the Related Art

Abnormal neovascularization or angiogenesis and enhanced vascular permeability are major causes for many ocular disorders including age-related macular degeneration (AMD), retinopathy of prematurity (ROP), ischemic retinal vein occlusions and diabetic retinopathy (DR). AMD and DR are among the most common cause of severe, irreversible vision loss. In these and related diseases, such as retinal vein occlusion, central vision loss is secondary to angiogenesis, the development of new blood vessels from pre-existing vasculature, and alterations in vascular permeability properties.

The angiogenic process is known by the activation of quiescent endothelial cells in pre-existing blood vessels. The normal retinal circulation is resistant to neovascular stimuli, and very little endothelial cell proliferation takes place in the retinal vessels. While there appear to be many stimuli for retinal neovascularization, including tissue hypoxia, inflammatory cell infiltration and penetration barrier breakdown, all increase the local concentration of cytokines (VEGF, PDGF, FGF, TNF, IGF etc.), integrins and proteinases resulting in the formation of new vessels, which then disrupt the organizational structure of the neural retina or break through the inner limiting membranes into the vitreous. Elevated cytokine levels can also disrupt endothelial cell tight junctions, leading to an increase in vascular leakage and retinal edema, and disruption of the organizational structure of the neural retina. Although VEGF is considered to be a major mediator of inflammatory cell infiltration, endothelial cell proliferation and vascular leakage, other growth factors, such as PDGF, FGF, TNF, and IGF etc., are involved in these processes. Therefore, growth factor inhibitors can play a significant role in inhibiting retinal damage and the associated loss of vision upon local delivery in the eye or via oral dosing.

There is no cure for the diseases caused by ocular neovascularization and enhanced vascular permeability. The current treatment procedures of AMD include laser photocoagulation and photodynamic theraphy (PDT). The effects of photocoagulation on ocular neovascularization and increased vascular permeability are achieved only through the thermal destruction of retinal cells. PDT usually requires a slow infusion of the dye, followed by application of non-thermal laser-light. Treatment usually causes the abnormal vessels to temporarily stop or decrease their leaking. PDT treatment may have to be repeated every three months up to 3 to 4 times during the first year. Potential problems associated with PDT treatment include headaches, blurring, and decreased sharpness and gaps in vision and, in 1-4% of patients, a substantial decrease in vision with partial recovery in many patients. Moreover, immediately following PDT treatment, patients must avoid direct sunlight for 5 days to avoid sunburn. Recently, a recombinant humanized IgG monoclonal antibody fragment (ranibizumab) was approved in the US for treatment of patients with age-related macular degeneration. This drug is typically administered via intravitreal injection once a month.

Many compounds that may be considered potentially useful in treating ocular neovascularization and enhanced vascular permeability-related and other disorders, are poorly soluble in water. A poorly water soluble compound is a substance that is not soluble at a therapeutically effective concentration in an aqueous physiologically acceptable vehicle. Aqueous solubility is an important parameter in formulation development of a poorly water soluble compound. What is needed is a formulation that provides increased solubility of the compound while also providing sufficient bioavailability of the compound so as to maintain its therapeutic potential.

The present invention provides safe and effective formulations for ocular administration of poorly soluble compounds for the treatment of ocular diseases caused by endothelial cell proliferation, vascular leakage, inflammation and angiogenesis.

### SUMMARY OF THE INVENTION

The present invention overcomes these and other drawbacks of the prior art by providing compositions for treating ocular diseases due to angiogenesis, enhanced endothelial cell proliferation, inflammation, or increased vascular permeability. Within one aspect of the present invention, a pharmaceutical composition is provided wherein a compound having poor water solubility is incorporated into cyclodextrin derivative in suitable buffer containing a nonionic surfactant, a dispersant and tonicity agent to develop an intraocular formulation for use in vitreoretinal therapy, in treating angiogenesis-related ocular disorders, inhibiting neovascularization, controlling vascular permeability, treating inflammation, and improving vision. The solubility of the compounds for use in the compositions of the present invention is substantially enhanced via incorporation of a cyclodextrin derivative into the composition. The compositions of the invention include an agent having poor water solubility and at least one cyclodextrin derivative.

The concentration of the anti-angiogenic, anti-inflammatory, or anti-vascular permeability agent used in this present invention varies depending on the ophthalmic diseases and the route of administration used, and any concentration may be employed as long as its effect is exhibited. Thus, although the concentration is not restricted, a concentration of 0.001% to 10 wt % is preferred. The concentration of cyclodextrin will vary depending on the concentration of active used in the formulation. Although the concentrations are not restricted, usually, the preferred concentration of the cyclodextrin derivative in the intravitreal composition is from 0.1% to 25%, more preferred concentration is 0.5% to 15%, and most preferred concentration is 1% to 10%.

In another embodiment, posterior juxtascleral (PJ) and periocular (PO) formulations containing (a) an active agent, such as an anti-angiogenic compound, an anti-inflammatory compound, or an anti-vascular permeability agent; (b) a suitable amount of a cyclodextrin derivative; (c) a suitable buffer; (d) tonicity agents in an amount such that tonicity is around 300 mOsm/kg; (e) a suspending agent; and (f) a surfactant are provided.

In yet another embodiment, the present invention provides formulations for topical ocular dosing, which include (a) a therapeutically effective amount of an active agent, such as an anti-angiogenic agent, an anti-inflammatory compound, or an anti-vascular permeability agent; (b) a suspending agent; (c) a surfactant; (d) tonicity agent; (d) cyclodextrin derivative; and (e) a buffer.

A wide variety of molecules may be utilized within the scope of present invention, especially those molecules having very low solubility. As used herein, the term “poor solubility” is used to refer to a compound having solubility in water or vehicle of less than 10 μg/mL, well below its therapeutic window. It is desirable to have a concentration of soluble drug in the formulation of at least 200 μg/mL for local ocular delivery to elicit desirable biological activities.

The compositions of the present invention are preferably administered to the eye of a patient suffering from an angiogenesis or enhanced vascular permeability related ocular, or a disorder characterized by neovascularization or vascular permeability, via posterior juxtascleral administration, intravitreal injection, topical ocular administration, or vitreoretinal therapy.

### BRIEF DESCRIPTION OF THE DRAWINGS

The following drawings form part of the present specification and are included to further demonstrate certain aspects of the present invention. The invention may be better understood by reference to these drawings in combination with the detailed description of specific embodiments presented herein.

FIG. 1 shows the effects of increasing concentrations of a cyclodextrin derivative, hydroxypropyl-β-cyclodextrin (HPCD), on the solubility of a receptor tyrosine kinase inhibitor (RTKi). More than 1800 fold of solubility increase is observed in presence of 10% HPCD.

FIG. 2 shows the effects of increasing concentrations of a cyclodextrin derivative, sulfobutylether-β-cyclodextyrin (SBCD), on the solubility of a receptor tyrosine kinase inhibitor (RTKi). More than 4200 fold solubility increased in presence of 10% SBCD.

FIG. 3 shows the effects of single intravitreal injection of a receptor tyrosine kinase inhibitor (1%) in vehicle containing Hydroxypropyl Cyclodextrin (HPCD) against preretinal neovascularization in the VEGF induced rat vascular leakage model. The formulation showed significant decrease of vascular leakage.

### DETAILED DESCRIPTION PREFERRED EMBODIMENTS

As noted above, the present invention provides compositions that contain an active agent having poor water solubility, for use in the treatment of ocular disorders caused by endothelial cell proliferation, enhanced vascular permeability, inflammation, or angiogenesis. The compositions of the invention are useful in treating disorders associated with microvascular pathology, increased vascular permeability and intraocular neovascularization, including diabetic retinopathy (DR), age-related macular degeneration (AMD) and retinal edema.

Briefly, within the context of the present invention, an active agent should be understood to be any molecule, either synthetic or naturally occurring, which acts to inhibit vascular growth, reduce vascular permeability, and/or decrease inflammation. In particular, the present invention provides compositions comprising an insoluble, or poorly soluble, active agent in a therapeutically effective amount solubilized into a cyclodextrin derivative for ophthalmic use.

Cyclodextrins are novel chemically stable, torus-shaped, circular and nonreducing oligosaccharides prepared by enzymatic degradation of starch. Cyclodextrins with lipophilic inner cavities and hydrophilic outer surfaces are easily water soluble and are capable of interacting with a variety of molecules to form non-covalent inclusion complexes. The size of the central cavity varies according to cyclodextrin type. Cyclodextrins are also known to exhibit other non bonding interactions with various molecules in solution. Molecular complexation or inclusion complex formation is dependent on the size of the molecule as well as the cavity size of the cyclodextrin.

Cyclodextrins have been used in certain pharmaceutical preparations to enhance the solubility and stability of a chemical entity. For example U.S. Pat. No. 4,727,064 describes the application of a cyclodextrin derivative to form inclusion complexes with improved solubility and dissolution properties. U.S. Pat. Nos. 5,024,998 and 4,983,586 disclose compositions comprising complexes of hydroxypropyl cyclodextrin (HPCD) with a difficult to solubilize drug where the amount of HPCD ranges from 20-50%. Preparation of compositions containing cyclodextrins to solubilize the poorly soluble compounds for use in the formulations of the present invention is within the knowledge of the skilled artisan (see. e.g., Rajeswari et al. 2005; Alberts and Muller 1995; Menard et al. 1990; Szejtili 1994; Loftsson and Stefansson 2002; and Rajewski and Stella 1996). Compositions including cyclodextrins were also reported in U.S. Pat. No. 6,232,343.

It is contemplated that any active agent that is poorly water soluble may be included in the compositions of the present invention. For example, anti-angiogenic agents, anti-inflammatory agents, or anti-vascular permeability agents are useful in the compositions of the invention.

Preferred anti-angiogenic agents include, but are not limited to, receptor tyrosine kinase inhibitors (RTKi), in particular, those having a multi-targeted receptor profile such as that described in further detail herein; angiostatic cortisenes; MMP inhibitors; integrin inhibitors; PDGF antagonists; antiproliferatives; HIF-1 inhibitors; fibroblast growth factor inhibitors; epidermal growth factor inhibitors; TIMP inhibitors; insulin-like growth factor inhibitors; TNF inhibitors; antisense oligonucleotides; etc. and prodrugs of any of the aforementioned agents. The preferred anti-angiogenic agent for use in the present invention is a multi-targeted receptor tyrosine kinase inhibitor (RTKi). Most preferred are RTKi's with multi-target binding profiles, such as N-[4-(3-amino-1H-indazol-4-yl) phenyl]-N′-(2-fluoro-5-methylphenyl) urea, having the binding profile substantially similar to that listed in Table 1. Additional multi-targeted receptor tyrosine kinase inhibitors contemplated for use in the compositions of the present invention are described in U.S. application Ser. No. 2004/0235892, incorporated herein by reference. As used herein, the term “multi-targeted receptor tyrosine kinase inhibitor” refers to a compound having a receptor binding profile exhibiting selectivity for multiple receptors shown to be important in angiogenesis, such as the profile shown in Table 1, and described in co-pending U.S. application Ser. No. 2006/0189608, incorporated herein by reference. More specifically, the preferred binding profile for the multi-targeted receptor tyrosine kinase inhibitor compounds for use in the compositions of the present invention is KDR (VEGFR2), Tie-2 and PDGFR.

TABLE 1
Kinase Selectivity Profile of a RTK Inhibitor

| KDR | FLT1 | FLT4 | PDGFR | CSF1R | KIT | FLT3 | TIE2 | FGFR | EGFR | SRC |
|---|---|---|---|---|---|---|---|---|---|---|
| 4 | 3 | 190 | 66 | 3 | 14 | 4 | 170 | >12,500 | >50,000 | >50,000 |

All data reported as IC50 values for kinase inhibition in cell-free enzymatic assays; ND denotes no data. Values determined @ 1 mM ATP.

Other agents which will be useful in the compositions and methods of the invention include anti-VEGF antibody (i.e., bevacizumab or ranibizumab); VEGF trap; siRNA molecules, or a mixture thereof, targeting at least two of the tyrosine kinase receptors having IC₅₀ values of less than 200 nM in Table 1; glucocorticoids (i.e., dexamethasone, fluoromethalone, medrysone, betamethasone, triamcinolone, triamcinolone acetonide, prednisone, prednisolone, hydrocortisone, rimexolone, and pharmaceutically acceptable salts thereof, prednicarbate, deflazacort, halomethasone, tixocortol, prednylidene (21-diethylaminoacetate), prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, and deacylcortivazol oxetanone); Naphthohydroquinone antibiotics (i.e., Rifamycin); and NSAIDs (i.e., nepafenac, amfenac).

Preferred cyclodextrin derivatives for use in the compositions of the present invention include alpha cyclodextrin, beta cyclodextrin, gamma cyclodextrin, dimethyl beta cyclodextrin, trimethyl beta cyclodextrin, hydroxyethyl beta cyclodextrin, hydroxypropyl gamma cyclodextrin, sulfated beta cyclodextrin, sulfated alpha cyclodextrin, beta cyclodextrin polymer, sulfobutyl ether beta cyclodextrin, glucosyl-cyclodextrin, maltosyl-cyclodextrin, quaternary ammonium beta cyclodextrin polymer and the like.

Hydroxypropyl-β-cyclodextrin (HPCD) is commercially available as a pyrogen free product. It is nonhygroscopic white powder that readily dissolves in water. HPCD is thermally stable and does not degrade at neutral pH. Chemical structure of a Cyclodextrin compound is shown below.

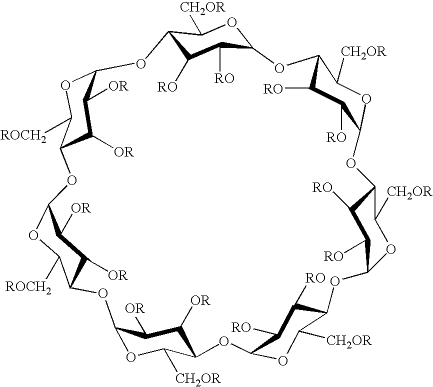

The formulations of the present invention provide a number of advantages over conventional formulations. One advantage of the present invention is that cyclodextrin derivatives can successfully solubilize poorly soluble compounds, allowing the preparation of an efficacious ophthalmologically acceptable intravitreal, PJ and/or periocular formulation for local ocular delivery. Additionally, bio availability of the drug can be modulated by controlling the amount and type of cyclodextrin derivative used in the formulation. Encapsulation of the compound by cyclodextrin derivatives can protect the compound from metabolic degradation upon local delivery. Furthermore, the preparation can be injected using a 27 or 30 gauge needle. Another advantage of the compositions of the present invention is that chemical stability of the active compound may be improved since the active compound is encapsulated within the cavity of the cylcodextrin compounds. Likewise, toxicity of the active compound can be reduced or suitably modulated.

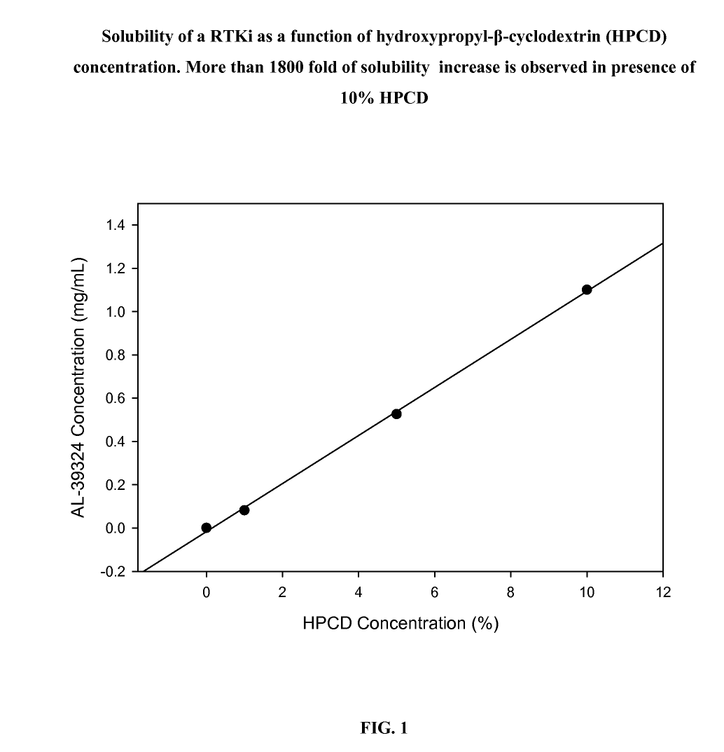

The present inventors have discovered that use of cyclodextrin derivatives to solubilize highly insoluble anti-angiogenic active compounds provides an efficacious ophthalmic formulation. For example, the compound N-[4-(3-amino-1H-indazol-4-yl) phenyl]-N′-(2-fluoro-5-methylphenyl) urea has extremely poor solubility in phosphate buffer, pH 7.2 (0.00059 mg/mL). Addition of 1%, 5% or 10% HPCD increased the solubility of the active compound significantly (Table 2, FIG. 1). At 10% HPCD concentration in phosphate buffer, the solubility of the active compound was 1.09 mg/mL, which corresponded to about 0.1%. Thus, approximately 1800 fold solubility enhancement of the active compound is accomplished by using 10% HPCD in phosphate buffer. Sulfobutylether-β-Cyclodextrin (SBCD) showed better solubilizing power, and at 10% SBCD in phosphate buffer the solubility of the RTKi compound was 2.52 mg/mL, which corresponded to 4200 fold increase of solubility (Table 3). Prototype intravitreal vehicle is shown in Example 1, and intravitreal and PJ formulations of the active compound containing HPCD are provided in Examples 2 and 3, respectively.

Solubility of RTKi in phosphate buffer vehicle containing various amount of hydroxypropyl cyclodextrin (HPCD). The solubility of the RTKi as a function of HPCD is is shown in FIG. 1.

TABLE 2

| | RTKi Solubility |
|---|---|
| Solution | (mg/mL) |
| Phosphate Buffer (0% HPCD) | 0.00059 |
| 1% HPCD in Phosphate Buffer | 0.0823 |
| 5% HPCD Phosphate Buffer | 0.5268 |
| 10% HPCD Phosphate Buffer | 1.0988 |

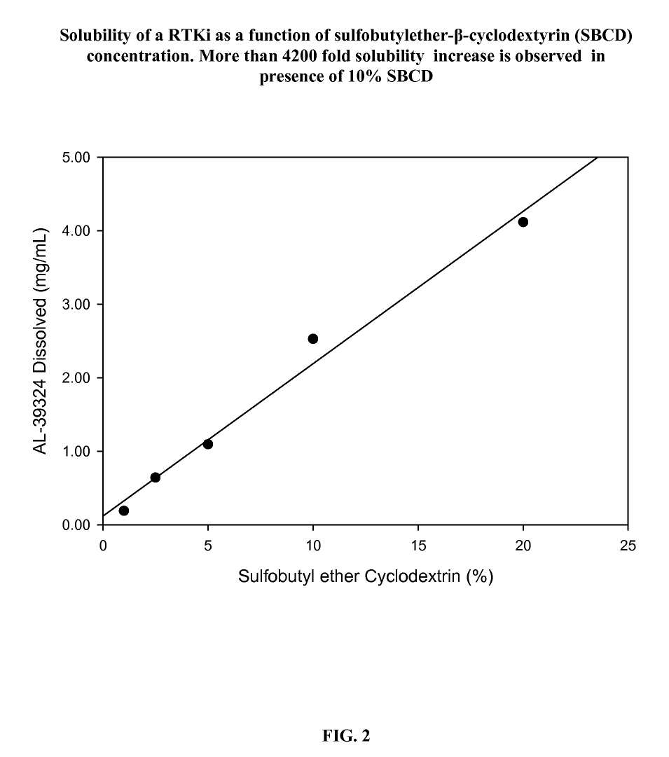

Solubility of RTKi in phosphate buffer vehicle containing various amount of sulfobutylether beta cyclodextrin (SBCD) is presented in Table 3. The solubility of the RTKi as a function of SBCD is shown in FIG. 2.

TABLE 3

| | RTKi Solubility |
|---|---|
| Solution | (mg/mL) |
| Phosphate Buffer (0% SBCD) | 0.00059 |
| 1% SBCD in Phosphate Buffer | 0.1881 |
| 2.5% SBCD Phosphate Buffer | 0.6406 |
| 5% SBCD Phosphate Buffer | 1.0940 |
| 10% SBCD Phosphate Buffer | 2.5265 |
| 20% SBCD Phosphate Buffer | 4.1152 |

While the preferred cyclodextrin derivative for use in the compositions of the present invention is HPCD, it is contemplated that other cyclodextrin derivatives may be used either alone or in combination. For example, cyclodextrin derivatives such as alpha cyclodextrin, beta cyclodextrin, gamma cyclodextrin, trimethyl beta cyclodextrin, hydroxyethyl beta cyclodextrin, hydroxypropyl gamma cyclodextrin, sulfated beta cyclodextrin, sulfated alpha cyclodextrin, beta cyclodextrin polymer, sulfobutyl ether beta cyclodextrin and quaternary ammonium beta cyclodextrin polymer.

In certain preferred embodiments, the formulation of the invention will further comprise a suitable viscosity agent, such as hydroxypropyl methylcellulose, hydroxyethyl cellulose, polyvinylpyrrolilidone, carboxymethyl cellulose, polyvinyl alcohol, sodium chondrointin sulfate, sodium hyaluronate etc. as a dispersant, if necessary. A nonionic surfactant such as polysorbate 80, polysorbate 20, tyloxapol, Cremophor, HCO 40 etc. can be used. The ophthalmic preparation according to the present invention may contain a suitable buffering system, such as phosphate, citrate, borate, tris, etc., and pH regulators such as sodium hydroxide and hydrochloric acid may also be used in the formulations of the inventions. Sodium chloride or other tonicity agents may be used to adjust tonicity, if necessary.

The specific dose level of the active agent for any particular human or animal depends upon a variety of factors, including the activity of the active compound used, the age, body weight, general health, time of administration, route of administration, and the severity of the pathologic condition undergoing therapy.

The formulations described herein may be delivered topically, via intravitreal injection, via posterior juxtascleral, and periocular routes. In preferred embodiments of the present invention, the amount of active agent, or poorly water soluble agent, will be from about 0.001% to 10% for intravitreal administration. More preferably from 0.05% to 3% and most preferably from 0.1% to 2%.

Due to the intended route of administration (IVT or PJ), it is very important that the particle size of the formulations must be small to accomplish good syringibality, as well as comfort. Suspensions with particle size from 1 μm-3μm are prepared by this compounding procedure. The prepared formulations (for IVT or PJ) exhibit excellent syringibility even when only 2 μL-10 μL of the formulation is injected in the eyes of the animals. A general composition of an ophthalmic formulation of RTKi is provided in Table 4.

TABLE 4
General Composition of RTKi Ophthalmic Formulation

| Ingredient | Amount (w/v, %) |
|---|---|
| RTKi | 0.01-10 |
| Polysorbate 80 | 0.01-1 |
| Hydroxypropyl-β-Cyclodextrin (HPCD) | 0.1-20 |
| Dibasic Sodium Phosphate, Dodecahydrate | 0-0.5 |
| Viscosity enhancer | 0-0.5 |
| Sdoium Chloride | 0-0.9 |
| Hydrochloric acid | q.s. to pH |
| Sodium Hydroxide | q.s. to pH |
| Water for Injection | q.s. to 100 |

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

This example illustrates the preparation of Intravitreal formulation vehicle containing hydroxypropyl-β cyclodextrin (HPCD).

| Ingredient | Amount (w/v, %) |
|---|---|
| Polysorbate 80 | 0.1 |
| Hydroxypropyl-β-Cyclodextrin | 10 |
| Dibasic Sodium Phosphate, Dodecahydrate | 0.18 |
| Viscosity enhancer | 0.05 |
| Sodium Chloride | 0.55 |
| Hydrochloric acid | q.s. to pH 7.2 |
| Sodium Hydroxide | q.s. to pH 7.2 |
| Water for Injection | q.s. to 100 |

In a 150 mL glass container, was added 9 g sterile 2% dibasic sodium phosphate, dodecahydrate solution. To it was added 10 g hydroxypropyl-β cyclodextrin and stirred for about 30 min. To it was added 5 g sterile 2% polysorbate 80 solution, 2.5 g of sterile 2% stock HPMC 2910 (E4M) solution and 11 g of 5% sterile sodium chloride solution, and stirred well until homogeneous. Sterile water for injection was added to get to 95% of batch size. The solution was stirred at RT for 30 min and pH was adjusted to 7.2. Finally, water for injection was added to get final batch of 100 g.

### EXAMPLE 2

This example illustrates the preparation of RTKi (N-[4-(3-amino-1H-indazol-4-yl) phenyl]-N′-(2-fluoro-5-methylphenyl) urea) Intravitreal Formulation containing hydroxypropyl-β cyclodextrin (HPCD).

| Ingredient | Amount (w/v, %) |
|---|---|
| RTKi | 1 |
| Polysorbate 80 | 0.1 |
| Hydroxypropyl-β-Cyclodextrin | 10 |
| Dibasic Sodium Phosphate, Dodecahydrate | 0.18 |
| Viscosity enhancer | 0.05 |
| Sodium Chloride | 0.55 |
| Hydrochloric acid | q.s. to pH 7.2 |
| Sodium Hydroxide | q.s. to pH 7.2 |
| Water for Injection | q.s. to 100 |

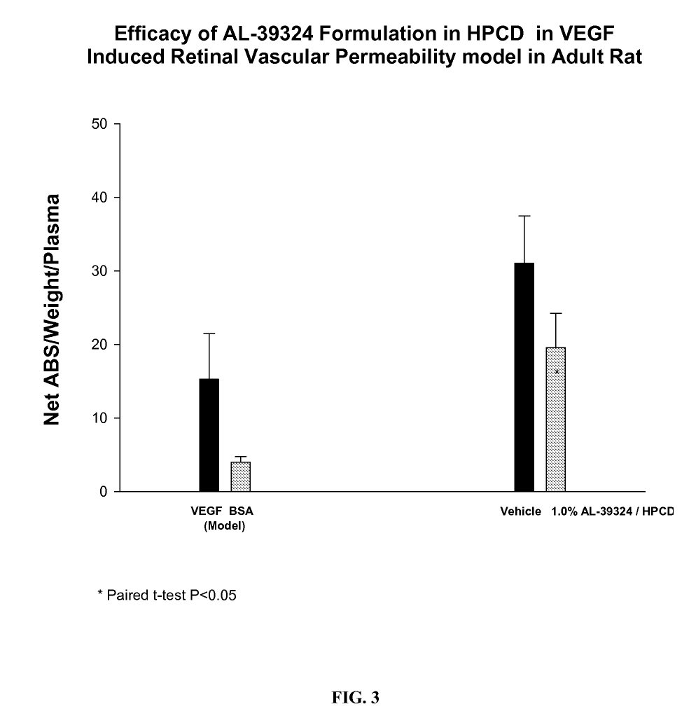

In a 250 mL glass container, carefully weigh 1 g sterile RTKi raw material. To it was added 20 g of 0.5% Polysorbate 80 solution. The suspension was ball milled for 8 h using Zirconia beads. Upon completion of ball milling, the suspension was filtered through a Buchner funnel; beads were washed thoroughly with water. To it was added 9 g sterile 2% dibasic sodium phosphate, dodecahydrate solution and 10 g hydroxypropyl-β cyclodextrin. The solution was stirred for about 30 min. To it were added 2.5 g of sterile 2% stock HPMC 2910 (E4M) solution and 11 g of 5% sterile sodium chloride solution, and stirred well until homogeneous. Sterile water for injection was added to get to 95% of batch size. The solution was stirred at RT for 30 min and pH was adjusted to 7.2. Finally, water for injection was added to get final batch of 100 g. The above formulation was intravitreally administered in VEGF induced rat vascular permeability model and the results are shown in FIG. 3.

### EXAMPLE 3

This example demonstrates the preparation of RTKi (N-[4-(3-amino-1H-indazol-4-yl) phenyl]-N′-(2-fluoro-5-methylphenyl) urea) formulation for PJ and/or periocular use.

| Ingredient | Amount (w/v, %) |
|---|---|
| RTKi | 3 |
| Polysorbate 80 | 0.3 |
| Hydroxypropyl-β-Cyclodextrin | 15 |
| Dibasic Sodium Phosphate, Dodecahydrate | 0.18 |
| Viscosity enhancer | 0.2 |
| Sodium Chloride | 0.5 |
| Hydrochloric acid | q.s. to pH 7.2 |
| Sodium Hydroxide | q.s. to pH 7.2 |
| Water for Injection | q.s. to 100 |

In a 250 mL glass container, carefully weigh 3 g sterile RTKi raw material. To it was added 30 g of 1% Polysorbate 80 solution. The suspension was ball milled for 8 h using Zirconia beads. Upon completion of ball milling, the suspension was filtered through a Buchner funnel; beads were washed thoroughly with water. To it was added 4.5 g sterile 4% dibasic sodium phosphate, dodecahydrate solution and 15 g hydroxypropyl-β cyclodextrin. The solution was stirred for about 30 min. To it were added 10 g of sterile 2% stock HPMC 2910 (E4M) solution and 11 g of 5% sterile sodium chloride solution, and stirred well until homogeneous. Sterile water for injection was added to get to 95% of batch size. The solution was stirred at RT for 30 min and pH was adjusted to 7.2. Finally, water for injection was added to get final batch of 100 g.

### EXAMPLE 4

Rat VEGF Model: Vascular endothelial growth factor (VEGF) is a potent vascular permeability factor and is upregulated in diabetic retinas harvested from human patients and animal models. VEGF is thought to play a primary role in the development of retinal microvascular permeability and subsequent macular edema (DME) and related diseases. Therefore, RTKi (N-[4-(3-amino-1H-indazol-4-yl) phenyl]-N′-(2-fluoro-5-methylphenyl) urea) in cyclodextrin formulation was evaluated in the rat model of VEGF-induced retinal vascular permeability. During local intravitreal administration studies, adult Sprague-Dawley rats were randomly assigned to treatment groups and generally received an intravitreal (10 μL) injection of drug or vehicle in both eye or drug in one eye and vehicle in the contralateral eye. At 72 hours following the treatment/vehicle injection, both eyes of each rat were challenged with an intravitreal injection of 500 ng hrVEGF. Twenty-four hours after injection of VEGF, intravenous infusion of Evans blue dye was performed, and after the dye had circulated for 90 minutes, rats were euthanized. Following euthanasia, both eyes were enucleated following systemic perfusion with buffered salt solution, and the retinas harvested. The degree of retinal vascular permeability for each retina was calculated using the mean ± s.e.m. of net ABS/wet weight/plasma ABS, which was used for statistical analyses; P≦0.05 was considered significant.

Cyclodextrin based formulations of RTKi (1%) upon intravitreal administration in VEGF induced rat vascular permeability model showed statistically significat reduction of vascular leakage in rat eyes as shown in FIG. 3. Vascular leakage caused by VEGF was controlled by RTKi in the cyclodextrin based formulation.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

United States Patents

U.S. Pat. No. 4,727,064

U.S. Pat. No. 5,024,998

U.S. Pat. No. 4,983,586

U.S. Pat. No. 6,232,343

Books
C. Rajeswari, A. Ahuja, J. Ali and R. K. Kar in AAPS PharmSci Tech, 2005, 6 (2), E329
E. Alberts and B. W. Muller, Critical Reviews in Therapeutic Drug carrier systems, 1995, 12(4), 311
F. A. Menard, M. G. Dedhiya and C. T. Rohdes, Drug Development and Industrial Pharmacy, 1990, 16(1), 91J.

Other Publications
J. Szejtili, Med. Res. Rev. 1994, 14, 353
T. Loftsson, M. E. Brewster, J. Pharm Sci. 1996,85,1017
R. A. Rajewski and V. J. Stella, J. Pharm. Sci. 1996, 85, 1142
J. Szejtli, Chem. Rev. 1998, 98, 1743
K. Uekama, F. Hirayama, and T. Irie, Chem. Rev. 1998, 98, 2045
T. Loftsson and T. Jarvinen, Adv Drug Delivery Rev. 1999, 36, 59
T. Loftsson and E. Stefansson, Acta Ophthalmologica Scandinavica, 2002, 80, 144

We claim:

1. A composition for treating ocular neovascularization, said composition comprising:
a poorly water soluble active agent in an amount of from 0.01% to 20%, wherein said anti-angiogenic agent is encapsulated in a Cyclodextrin derivative.

2. The ophthalmic composition of claim 1, wherein the active agent is selected from the group consisting of anti-angiogenic agents, anti-inflammatory agents, and anti-vascular permeability agents.

3. The ophthalmic composition of claim 2, wherein the active agent is an anti-angiogenic agent.

4. The ophthalmic composition of claim 3, wherein the anti-angiogenic agent is a multi-targeted receptor tyrosine kinase (RTK) inhibitor.

5. The ophthalmic composition of claim 4, wherein the RTK inhibitor is N-[4-(3-amino-1H-indazol-4-yl) phenyl]-N′-(2-fluoro-5-methylphenyl) urea.

6. The ophthalmic composition of claim 1, wherein the said concentration of the anti-angiogenic agent is from 1% to 10%.

7. The ophthalmic composition of claim 5, wherein the said concentration of the anti-angiogenic agent is from 1% to 10%.

8. The composition of claim 1, comprising a β-Cyclodextrin derivative.

9. The ophthalmic composition of claim 8, wherein the β-Cyclodextrin compound is Hydroxypropyl-β-Cyclodextrin (HPCD).

10. The ophthalmic composition of claim 9, wherein the concentration of HPCD in the formulation is from 1% to 20%.

11. A composition for intravitreal injection for the treatment of ocular neovascularization, said composition comprising from 0.1 to 5% of a multi-targeted receptor tyrosine kinase inhibitor encapsulated in a Cyclodextrin derivative.

12. A composition for posterior juxtascleral and periocular injection for the treatment of ocular neovascularization, said composition comprising from 0.5 to 10% of a multi-targeted receptor tyrosine kinase inhibitor encapsulated in a Cyclodextrin derivative.

13. The composition of claim 11, wherein the RTK inhibitor is N-[4-(3-amino-1H-indazol-4-yl) phenyl]-N′-(2-fluoro-5-methylphenyl) urea.

14. The composition of claim 12, wherein the RTK inhibitor is N-[4-(3-amino-1H-indazol-4-yl) phenyl]-N′-(2-fluoro-5-methylphenyl) urea.
